(19) United States
(12) Patent Application Publication
(10) Pub. No.: US 2024/0148364 A1
(43) Pub. Date: May 9, 2024

(54) ULTRASOUND IMAGE PROCESSING APPARATUS, ULTRASOUND IMAGE DIAGNOSIS SYSTEM, ULTRASOUND IMAGE PROCESSING METHOD, AND NON-TRANSITORY COMPUTER-READABLE RECORDING MEDIUM STORING ULTRASONIC IMAGE PROCESSING PROGRAM

(71) Applicant: Konica Minolta, Inc., Tokyo (JP)

(72) Inventors: Hiroaki MATSUMOTO, Kanagawa (JP); Shikou KANEKO, Saitama (JP); Yoshihiro TAKEDA, Tokyo (JP)

(21) Appl. No.: 18/494,371

(22) Filed: Oct. 25, 2023

(30) Foreign Application Priority Data
Nov. 4, 2022 (JP) 2022-177396

(51) Int. Cl. A61B 8/08; A61B 8/00

(52) U.S. Cl. CPC A61B 8/5207; A61B 8/4461; A61B 8/461; A61B 8/48

(57) ABSTRACT

An ultrasound image processing apparatus includes a reception section that receives an ultrasound image and discriminator determination information from an external apparatus, the ultrasound image being generated based on a reception signal acquired by an ultrasound probe that transmits and receives an ultrasound wave to and from a subject, the discriminator determination information allowing determination of a discriminator that discriminates a target in the ultrasound image, and one or more first hardware processors, in which the one or more first hardware processors determine one from among a plurality of discriminators to which the ultrasound image is to be input, the one discriminator being determined based on the discriminator determination information, input the ultrasound image to the determined discriminator to acquire a discrimination result to be output from the determined discriminator.

### CROSS REFERENCE TO RELATED APPLICATIONS

The entire disclosure of Japanese Patent Application No. 2022-177396 filed on Nov. 4, 2022 is incorporated herein by reference in its entirety.

### BACKGROUND

### Technological Field

The present invention relates to an ultrasound image processing apparatus, an ultrasound image diagnostic system, an ultrasound image processing method, and a non-transitory computer-readable recording medium storing an ultrasound image processing program.

### Description of Related Art

There has been proposed an ultrasonographic device that, when a doctor or a laboratory technician performs an ultrasound examination using the ultrasonographic device, uses a trained model trained using machine learning to automatically recognize a region of interest or a cross-section determination in an image, thereby reducing a work load during measurement and assisting in understanding anatomy.

For example, in Japanese Unexamined Patent Publication No. 2021-164573, first ultrasonic data based on B-mode data acquired by transmitting and receiving ultrasound waves to and from the carotid artery of a subject is acquired. Then, the detection section detects the intima-media thickness of the carotid artery in the first ultrasound data by inputting the first ultrasound data to a trained model trained using the intima-media thickness of the carotid artery in each of a plurality of second ultrasound data and a plurality of second ultrasound data.

Incidentally, different types of ultrasound probes can be connected to the ultrasonographic device. The acquired ultrasound image has significantly different image quality and image features for each ultrasound probe. For example, a sector probe used in heart examination and a convex probe used in abdomen examination have different imaging ranges. In obstetrics, an intracavitary probe used in transvaginal examination and a convex probe used in transabdominal examination have different imaging ranges.

There has been a problem that, in a case where a trained model trained with an ultrasound image of a specific ultrasound probe is used and automatic recognition on an ultrasound image acquired with a different ultrasound probe is performed, accuracy of automatic recognition decreases because the trained model has not learned image features of the different ultrasound probe.

In addition, in recent years, remote ultrasound diagnosis has been performed in which automatic recognition is performed on an ultrasound image acquired by an ultrasonographic device, a moving image and an automatic recognition result are transferred through the internet, and measurement and diagnosis are performed by another image display apparatus. In particular, since a visit by a doctor or a laboratory technician cannot take place in the COVID-19 pandemic, there is a tendency that the number of cases where capturing of an ultrasound image is assigned to an on-site medical staff and a diagnosis is to a remote doctor increases.

In the case of on-site diagnostics and telemedicine, there is often not enough on-site equipment available, and a portable ultrasonographic device, which is simple and immediately usable, is often used according to the condition of a patient. In this case, functions used for diagnosis may be insufficient, or models (manufacturers, types of ultrasound probes, and the like) to be used may vary depending on the site.

Therefore, a doctor needs to perform remote ultrasonic diagnosis under various environments, for example, using images of a plurality of types of ultrasonographic devices. For an image acquired by an ultrasonographic device having no automatic recognition function, measurement and diagnosis are performed without referring to the automatic recognition result. In addition, in a case where automatic recognition is performed using the same trained model regardless of the type of ultrasound probe, teacher data suitable for the ultrasound probe being used may be yet to be leaned, and thus the accuracy of automatic recognition may be low. As a result, since automatic measurement cannot be performed, it is necessary for a doctor or a laboratory technician to manually perform measurement, which may increase time and effort or cause a problem in accuracy.

### SUMMARY

An object of the present invention is to provide an ultrasound image processing apparatus, an ultrasound image diagnostic system, an ultrasound image processing method, and a non-transitory computer-readable recording medium storing an ultrasound image processing program, which can perform automatic recognition more appropriately even when remote ultrasonic diagnosis is performed using a different ultrasound probe and/or ultrasonographic device.

An ultrasound image processing apparatus reflecting an aspect of the present invention in order to achieve at least one of the aforementioned objects includes:
a reception section that receives an ultrasound image and discriminator determination information from an external apparatus, the ultrasound image being generated based on a reception signal acquired by an ultrasound probe that transmits and receives an ultrasound wave to and from a subject, the discriminator determination information allowing determination of a discriminator that discriminates a target in the ultrasound image; and
one or more first hardware processors, in which
the one or more first hardware processors determine, from among a plurality of the discriminators, a discriminator to which the ultrasound image is to be input, the discriminator being determined based on the discriminator determination information received from the external apparatus, and
the one or more first hardware processors input the ultrasound image to the determined discriminator to acquire a discrimination result output from the determined discriminator

An ultrasound image diagnostic system reflecting an aspect of the present invention in order to achieve at least one of the aforementioned objects is
an ultrasound image diagnostic system, including:
an ultrasound image acquisition apparatus; and
an ultrasound image processing apparatus, in which:
the ultrasound image acquisition apparatus includes:
   an ultrasound probe that transmits and receives an ultrasound wave to and from a subject,
   a generation section that generates an ultrasound image based on a reception signal acquired by the ultrasound probe, and
   a transmission section that transmits, to the ultrasound image processing apparatus, the ultrasound image and discriminator determination information allowing determination of a discriminator that discriminates a target in the ultrasound image,
the ultrasound image processing apparatus includes:
   a reception section that receives the ultrasound image and the discriminator determination information,
   a plurality of discriminators that discriminate the target in the ultrasound image, and
   one or more first hardware processors, in which
the one or more first hardware processors determine, from among the plurality of the discriminators, a discriminator to which the ultrasound image is to be input, the discriminator being determined based on the discriminator determination information, and
the one or more first hardware processors input the ultrasound image to the determined discriminator to acquire a discrimination result output from the determined discriminator.

An ultrasound image processing method reflecting an aspect of the present invention in order to achieve at least one of the above-described objects includes:
receiving an ultrasound image and discriminator determination information from an external apparatus, the ultrasound image being generated based on a reception signal acquired by an ultrasound probe that transmits and receives an ultrasound wave to and from a subject, the discriminator determination information allowing determination of a discriminator that discriminates a target in the ultrasound image;
determining, from among a plurality of the discriminators, a discriminator to which the ultrasound image is to be input, the discriminator being determined based on the discriminator determination information received from the external apparatus; and
input the ultrasound image to the determined discriminator to acquire a discrimination result output from the determined discriminator.

A non-transitory computer-readable recording medium storing an ultrasound image processing program, which reflects an aspect of the present invention in order to achieve at least one of the aforementioned objects, is a non-transitory computer-readable recording medium storing an ultrasound image processing program for causing a computer to execute:
a process of receiving an ultrasound image and discriminator determination information from an external apparatus, the ultrasound image being generated based on a reception signal acquired by an ultrasound probe that transmits and receives an ultrasound wave to and from a subject, the discriminator determination information allowing determination of a discriminator that discriminates a target in the ultrasound image,
a process of determining, from among a plurality of the discriminators, a discriminator based on the discriminator determination information received from the external apparatus, the discriminator being one to which the ultrasound image is to be input, and
a process of inputting the ultrasound image to the determined discriminator to acquire a discrimination result output from the determined discriminator.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a block diagram illustrating an example of an ultrasound image diagnostic system according to an embodiment of the present invention;

FIG. 2 is a flowchart illustrating an example of an ultrasound image processing method in the ultrasound image diagnostic system illustrated in FIG. 1;

FIG. 3 is a diagram showing a selection screen for selecting an ultrasound probe on a display section of an ultrasound image acquisition apparatus shown in FIG. 1;

FIG. 4 is a view illustrating an ultrasound image of teacher data acquired using a sector probe, and an IVC region and a hepatic vein position to be automatically recognized by a discriminator; and

FIG. 5 is a view illustrating an ultrasound image of teacher data acquired using a convex probe, and an IVC region and a hepatic vein position to be automatically recognized by a discriminator.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, embodiments of the present invention will be described in detail with reference to the drawings.

[Ultrasound Image Diagnostic System]

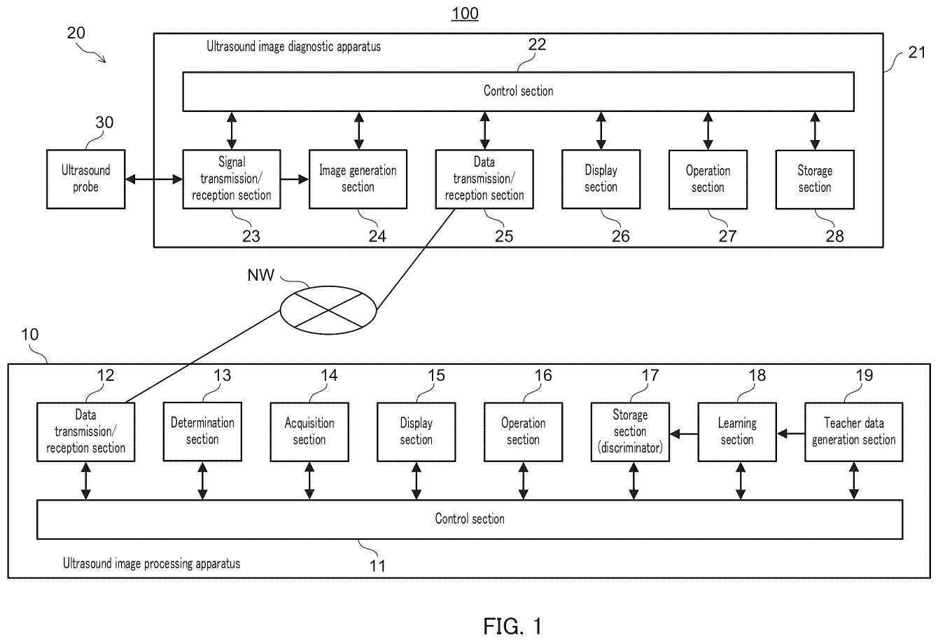

FIG. 1 is a block diagram illustrating an example of an ultrasound image diagnostic system 100 according to the present embodiment.

The ultrasound image diagnostic system 100 includes an ultrasound image processing apparatus 10 and an ultrasound image diagnostic apparatus 20 (ultrasound image acquisition apparatus in the present invention and external apparatus).

The ultrasound image processing apparatus 10 and the ultrasound image diagnostic apparatus 20 are communicably connected to each other via a network NW. As the network NW, the internet, a local area network (LAN), a wide area network (WAN), or the like is used, and wireless communication or wired communication can be used for the communication.

[Ultrasound Image Diagnostic Apparatus (Ultrasound Image Acquisition Apparatus)]

The ultrasound image diagnostic apparatus 20 transmits and receives ultrasound waves to and from a subject such as a living body, generates and displays the shape, property, or dynamics of biological tissue inside the subject as an ultrasound image based on reception signals acquired from the received ultrasound echoes, and is used for image diagnosis by a doctor or the like.

As illustrated in FIG. 1, the ultrasound image diagnostic apparatus 20 includes an apparatus body 21, an ultrasound probe 30, and the like. In the ultrasound image diagnostic apparatus 20, the ultrasound probe 30 is connected to the apparatus body 21 via a cable (not illustrated).

[Ultrasound Probe]

Although not shown in detail, the ultrasound probe 30 includes a plurality of transducers formed of piezoelectric elements. A plurality of transducers are arranged, for example, in a one dimensional array in a scanning direction of ultrasound waves. The ultrasound transducers may be arranged in a two dimensional array. In the ultrasound probe 30, the number of transducers can be arbitrarily set.

In the ultrasound probe 30, each transducer converts a drive signal output from the signal transmission/reception section 23 in the apparatus body 21, which will be described later, into an ultrasound wave, transmits the ultrasound wave to the inside of the subject, receives an ultrasound echo reflected in the subject, converts the ultrasound echo into a reception signal, and outputs the reception signal to the signal transmission/reception section 23.

As the ultrasound probe 30, various types of probes including, for example, a sector probe of a sector scanning type, a convex probe of a convex scanning type, a linear probe of a linear scanning type, an intracavitary probe, and the like can be used.

In the present embodiment, as described later, a type of the ultrasound probe 30 to be connected to the apparatus body 21 for use is selected when measurement is performed with the ultrasound image diagnostic apparatus 20. The connection between the apparatus body 21 and the ultrasound probe 30 may be performed by wireless communication such as UWB (Ultra Wide Band) instead of wired communication using a cable.

[Apparatus Body]

As shown in FIG. 1, the apparatus body 21 includes a control section 22, a signal transmission/reception section 23, an image generation section 24, a data transmission/reception section 25, a display section 26, an operation section 27, a storage section 28, and the like. As the apparatus body 21, a portable type, for example, a portable terminal such as a tablet-type terminal may be used.

The control section 22 has one or more hardware processors (the first hardware processors in the present invention). More specifically, the control section 22 includes a central processing unit (CPU) as an arithmetic/control device, a read only memory (ROM) and a random access memory (RAM) as a main storage device, and the like.

In the control section 22, the ROM stores programs and setting data in a non-transitory manner, and the CPU reads programs corresponding to processing contents from the ROM, loads the programs in the RAM that temporarily stores the programs, and executes the loaded programs. Thus, the control section 22 centrally controls the operation of each functional block of the ultrasound image diagnostic apparatus 20. That is, the control section 22 performs overall control on the ultrasound image diagnostic apparatus 20 by controlling each of the signal transmission/reception section 23, the image generation section 24, the data transmission/reception section 25, the display section 26, the operation section 27, and the storage section 28 according to their functions.

The signal transmission/reception section 23 includes a transmission section and a reception section (not shown). The transmission section supplies a drive signal, which is an electric signal, to the ultrasound probe 30 under the control of the control section 22, and causes the ultrasound probe 30 to generate an ultrasound wave.

Although detailed illustration is omitted, the transmission section includes, for example, a clock generation circuit, a delay circuit, and a pulse generation circuit. The clock generation circuit is a circuit that generates a clock signal that determines the transmission timing and the transmission frequency of the drive signal. The delay circuit is a circuit for setting a delay time for each individual path corresponding to each transducer of the ultrasound probe 30, delaying transmission of a drive signal by the set delay time, and performing focusing (transmission beam forming) of a transmission beam formed by transmission ultrasound waves. The pulse generation circuit is a circuit for generating a pulse signal as the drive signal at a set voltage and at a set time interval.

According to the control of the control section 22, the transmission section configured as described above performs scanning by sequentially switching a predetermined number transducers while shifting the transducers for each transmission or reception of the ultrasound wave and by supplying the drive signal to the plurality of transducers selected for output.

In the signal transmission/reception section 23, the reception section receives a reception signal, which is an electric signal, from the ultrasound probe 30 under the control of the control section 22.

Although detailed illustration is omitted, the reception section includes, for example, an amplifier, an A/D conversion circuit, and a phasing addition circuit. The amplifier is a circuit for amplifying the reception signal at a preset amplification rate for each individual path corresponding to each transducer. The A/D conversion circuit is a circuit for performing analog-digital conversion (A/D conversion) on the amplified reception signal. The phasing addition circuit is a circuit for giving a delay time to the A/D converted reception signal for each individual path corresponding to each transducer to adjust the time phase, and adding these (phasing addition) to generate sound ray data. That is, the phasing addition circuit performs reception beam forming on the reception signal of each transducer to generate sound ray data.

Under the control of the control section 22, the image generation section 24 performs envelope detection processing, logarithmic compression, and/or the like on the sound ray data from the reception section of the signal transmission/reception section 23, and performs brightness modulation by adjusting the dynamic range and the gain. As a result, the image generation section 24 generates B-mode image data (to be referred to as ultrasound image data hereinafter) as two dimensional tomographic image data. That is, the ultrasound image data represents the intensity of the reception signal by brightness. Note that the image generation section 24 may generate A-mode image data, M-mode image data (two dimensional tomographic image data), Doppler image data, color mode image data, or three dimensional image data.

In addition, the image generation section 24 includes an image memory section (not illustrated) which is configured by a semiconductor memory such as a dynamic random access memory (DRAM). The image generation section 24 causes the image memory section to store the generated ultrasound image data in units of frames.

Further, the image generation section 24 performs image processing such as image filter processing and time smoothing processing on the ultrasound image data read out from the image memory section, and performs scan conversion into a display image pattern to be displayed on the display section 26.

The data transmission/reception section 25 (transmitter in the invention) transmits the discriminator determination information to the ultrasound image processing apparatus 10 together with the ultrasound image data generated by the image generation section 24 under the control of the control section 22. In addition, the data transmission/reception section 25 receives a discrimination result acquired by processing using the discriminator in the ultrasound image processing apparatus 10. The received discrimination result is displayed on the display section 26 together with the ultrasound image.

The discriminator, which will be described in detail later, is for discrimination of a target being a discrimination target in the ultrasound image data. The discriminator determination information is information which allows determination of the discriminator, and is, for example, information on the type of the ultrasound probe 30. The discrimination result is, for example, a classification result of a measurement item, an automatic measurement result, a region of interest, or the like, and the workload of the operator is reduced by automatically acquiring such a result.

The display section 26 is a display device such as an LCD (Liquid Crystal Display), a CRT (Cathode-Ray Tube) display, an organic EL (Electronic Luminescence) display, an inorganic EL display, or a plasma display. The display section 26 displays an ultrasound image corresponding to the ultrasound image data generated by the image generation section 24 on the display screen under the control of the control section 22.

The operation section 27 includes various switches, buttons, a track pad, a track ball, a mouse, a keyboard, a touch panel that is integrally provided on the display screen of the display section 26 and detects a touch operation on the display screen, and the like.

The operation section 27 performs, for example, operation inputs such as inputs of selection of the type of the ultrasound probe 30, connection/disconnection of the ultrasound probe 30, selection of a diagnostic site, start/end of diagnosis, selection of a measurement item, start/end of a device or an application, and start/release of freezing. Furthermore, the operation section 27 inputs data such as personal information on the subject and various parameters for displaying an ultrasound image on the display section 26. The operation section 27 outputs an operation signal corresponding to the input to the control section 22.

Measurement items to be selected via the operation section 27 are morphological measurement using ultrasound image data (for example, length, area, angle, speed, and volume), measurement using a brightness value (for example, histogram), cardiac measurement, gynecological measurement, obstetric measurement, and the like. Note that in the present embodiment, by automatic recognition using a discriminator which will be described later, measurement on some of the measurement items is automatically performed without selection by the operation section 27.

The storage section 28 is a storage device capable of writing and reading information, such as a flash memory, a hard disk drive (HDD), or a solid state drive (SSD). The storage section 28 stores, for example, the ultrasound image data generated by the image generation section 24.

The signal transmission/reception section 23, the image generation section 24, the data transmission/reception section 25, and the like described above are configured by, for example, dedicated or general-purpose hardware (electronic circuit) corresponding to each process, and implement functions in cooperation with the control section 22.

For example, the signal transmission/reception section 23, the image generation section 24, the data transmission/reception section 25, and the like are configured by hardware of an integrated circuit such as a large scale integration (LSI). In addition, these sections may be configured by hardware such as a programmable logic device (PLD) including an application specific integrated circuit (ASIC), a digital signal processor (DSP), a field programmable gate array (FPGA), and/or the like. Alternatively, a reconfigurable processor in which connections and settings of circuit cells in an FPGA or an LSI can be reconfigured may be used.

[Ultrasound Image Processing Apparatus]

As illustrated in FIG. 1, the ultrasound image processing apparatus 10 includes a control section 11, a data transmission/reception section 12, a determination section 13, an acquisition section 14, a display section 15, an operation section 16, a storage section 17, a learning section 18, a teacher data generation section 19, and the like. The ultrasound image processing apparatus 10 may be an ultrasound image diagnostic apparatus similar to the ultrasound image diagnostic apparatus 20, and in this case, has the same configuration as the signal transmission/reception section 23 and the image generation section 24.

The control section 11 includes one or more hardware processors (the second hardware processors in the present invention). More specifically, the control section 11 includes a CPU as an arithmetic/control device, a ROM and a RAM as a main storage device, and the like.

The ROM is configured by a non-volatile memory such as a semiconductor, and stores, in a non-transitory manner, a system program corresponding to the ultrasound image processing apparatus 10, various processing programs executable on the system program, various data such as a lookup table (gamma correction), and the like. These programs are stored in the form of computer-readable program codes, and the CPU sequentially executes operations in accordance with the program codes. The RAM forms a work area for temporarily storing various programs to be executed by the CPU and data related to these programs. In the present embodiment, an “ultrasound image processing program” for executing an ultrasound image processing method described later is stored in the ROM of the control section 11.

As will be described in the ultrasound image processing method to be described later, the control section 11 performs automatic recognition on ultrasound image data using ultrasound image data and discriminator determination information transmitted from the ultrasound image diagnostic apparatus 20, acquires a discrimination result, and transmits the acquired discrimination result to the ultrasound image diagnostic apparatus 20.

The data transmission/reception section 12 (the receiver in the present invention) receives the ultrasound image data and the discriminator determination information transmitted from the ultrasound image diagnostic apparatus 20 and transmits the discrimination result acquired by the ultrasound image processing apparatus 10 under the control of the control section 11.

Under the control of the control section 11, the determination section 13 determines, based on the discriminator determination information received from the ultrasound image diagnostic apparatus 20, a discriminator to which the ultrasound image data is to be input, from among a plurality of discriminators for discriminating the discrimination target in the ultrasound image data.

For example, in a case in which the discriminator determination information indicates the type of the ultrasound probe 30, the determination section 13 determines a discriminator to which the ultrasound image data is to be input, from among a plurality of discriminators according to the type of the ultrasound probe 30.

Under the control of the control section 11, the acquisition section 14 inputs the ultrasound image data to the discriminator to acquire a discrimination result output from the discriminator.

The display section 15 is a display device such as an LCD, a CRT display, an organic EL display, an inorganic EL display, or a plasma display. Under the control of the control section 11, the display section 15 displays, on a display screen, an ultrasound image corresponding to the ultrasound image data transmitted from the ultrasound image diagnostic apparatus 20, the discriminator determination information, the discrimination result acquired by the acquisition section 14, and the like.

The operation section 16 includes various switches, buttons, a track pad, a track ball, a mouse, a keyboard, a touch panel that is integrally provided on the display screen of the display section 15 and detects a touch operation on the display screen, and the like. The operation section 16 performs, for example, operation/input on a display screen, operation input such as activation/termination of a device or an application, and the like. The operation section 16 outputs an operation signal corresponding to the input to the control section 11.

The storage section 17 is a storage device capable of writing and reading information, such as a flash memory, an HDD, or an SSD. The storage section 17 stores, for example, the ultrasound image data and discriminator determination information transmitted from the ultrasound image diagnostic apparatus 20, and the discrimination result acquired by the acquisition section 14.

In the present embodiment, the storage section 17 stores a plurality of discriminators used by the acquisition section 14 to discriminate a discrimination target captured in an ultrasound image transmitted from the ultrasound image diagnostic apparatus 20.

The discrimination target to be discriminated by the discriminator is, for example, a nerve, a fascia, a muscle, a blood vessel, a needle, a heart, a placenta, a lymph node, a brain, a prostate, a carotid artery or a breast. The discrimination target is not limited to a particular structure, and may be an organ itself, a body structure such as a hand, a foot, a face, a neck, or a waist, a lesion portion indicating some disease, or an abnormal brightness region in an ultrasound image. The storage section 17 may store a plurality of discriminators optimized for respective discrimination targets so as to enhance the accuracy of discriminating the discrimination target.

Under the control of the control section 11, the learning section 18 performs learning (machine learning) using the teacher data, thereby causing the discriminator to grasp the characteristics of the probability distribution latent in the teacher data. A trained discriminator typically becomes able to discriminate an image pattern simply by an input of pixel value information of ultrasound image data.

Specifically, the ultrasound image data transmitted from the ultrasound image diagnostic apparatus 20 is input to the discriminator. The discriminator is trained in advance to output, for each of predetermined portions constituting an ultrasound image corresponding to the input ultrasound image data, a confidence level indicating likelihood that a discrimination target (specifically, a position, a boundary, or a region of the discrimination target) appears in the ultrasound image. The confidence level output from the discriminator is represented by a value larger than 0 and equal to or smaller than 1. A higher confidence level means higher likelihood that the discrimination target appears in the ultrasound image.

Training for such a discriminator is performed by known supervised learning (specifically by adjusting network parameters including a weight coefficient, a bias, and the like using backpropagation) based on teacher data prepared in advance.

The teacher data generation section 19 generates teacher data to be learned by the learning section 18 for the discriminator under the control of the control section 11.

The teacher data is a set of ultrasound image data generated in the past and correct answer data corresponding thereto. As the correct answer data, for example, a labeling image acquired by labeling a desired region in the ultrasound image corresponding to the ultrasound image data with an arbitrary value, coordinate data indicating the desired region by coordinates, or an expression of a straight line or a curve indicating the boundary of the desired region is used.

In this embodiment, the labeling image is used as the correct answer data, and in the ultrasound image data as the teacher data, a portion in which a discrimination target appears is labeled with “1,” and a portion in which the discrimination target does not appear is labeled with “0.”

Note that examples of the above-described discriminator include a Convolutional Neural Network (hereinafter referred to as “CNN”).

The CNN is a type of forward-propagation neural network and is based on knowledge about the structure of the visual area of the brain. Basically, the CNN has a structure in which a convolution layer for extracting a local feature of an image and a pooling layer (subsampling layer) for collecting features for each local area are repeated.

Each of the layers of the CNN has a plurality of neurons, and the individual neurons are arranged so as to correspond to the visual area. The basic operation of each neuron consists of signal input and output. However, in a case where signals are transmitted between neurons in each layer, an input signal is not output as it is, but connecting weights are set respectively for inputs and a signal is output to a neuron in the next layer in a case where the sum of the weighted inputs exceeds a threshold value set for each neuron. The connecting weights between the neurons are calculated from the teacher data. Accordingly, an output value can be estimated by inputting real-time data.

Known CNN models include, for example, GoogleNet, ResNet, SENet, U-Net, and MobileNet, but algorithms forming a CNN are not particularly limited as long as the CNN is suitable for this purpose. The discriminator is also not limited to the CNN, and a mathematical model including a calculation algorithm or a coefficient may be used.

Further, the storage section 17 may include a plurality of types of discriminators having different characteristics. For example, the storage section 17 may have two types of discriminators different in confidence level. For example, one is a discriminator that has been learned to output a confidence level indicating the likelihood that a plurality of discrimination targets (corresponding to a plurality of general-purpose parts (classes)) appear in input ultrasound image data. The other is a discriminator that has been trained to output a confidence level indicating the likelihood that a specific discrimination target (corresponding to a specific part) appears in the input ultrasound image data.

Further, for example, the storage section 17 may include two types of discriminators having different responsiveness and discrimination accuracy. For example, as the discriminator used by the acquisition section 14 to discriminate the same discrimination target, one is a discriminator of a responsiveness-oriented model which places higher importance on responsiveness (discrimination processing time and real-time performance) than that on discrimination accuracy. The other is a discriminator of a discrimination accuracy-oriented model that places higher importance on discrimination accuracy than that on responsiveness.

Note that some or all of the functions of the data transmission/reception section 12, the determination section 13, the acquisition section 14, the learning section 18, and the teacher data generation section 19 may be implemented by dedicated or general-purpose hardware (electronic circuits) corresponding to the functions. In this case, the data transmission/reception section 12, the determination section 13, the acquisition section 14, the learning section 18, and the teacher data generation section 19 implement functions in cooperation with the control section 11.

For example, the data transmission/reception section 12, the determination section 13, the acquisition section 14, the learning section 18, and the teacher data generation section 19 are configured by hardware of an integrated circuit such as a large scale integration (LSI). In addition, these sections may be configured by hardware such as PLD including ASIC, DSP, FPGA, and the like. Alternatively, a reconfigurable processor in which connections and settings of circuit cells in an FPGA or an LSI can be reconfigured may be used.

On the other hand, a part or all of the functions of the data transmission/reception section 12, the determination section 13, the acquisition section 14, the learning section 18, and the teacher data generation section 19 may be executed by software. In this case, the software is stored in one or more storage media such as a ROM, an optical disk, a hard disk, or the like, and the software is executed by the CPU of the control section 11.

The ultrasound image processing apparatus 10 does not necessarily include the learning section 18 and the teacher data generation section 19. For example, an external apparatus including the learning section 18 and the teacher data generation section 19 may perform learning to generate a discriminator, and the discriminator generated by the external apparatus may be stored in the storage section of the ultrasound image processing apparatus 10 via a network.

In the present embodiment, the ultrasound image diagnostic system 100 uses the ultrasound image processing apparatus 10 and the ultrasound image diagnostic apparatus 20 having the above-described configurations, and performs automatic recognition more appropriately by a method described below.

[Ultrasound Image Processing Method]

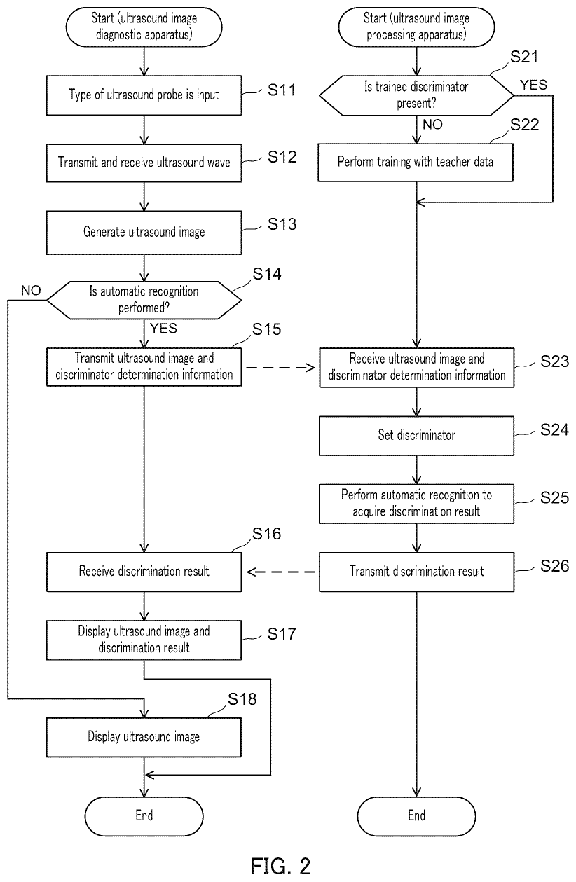

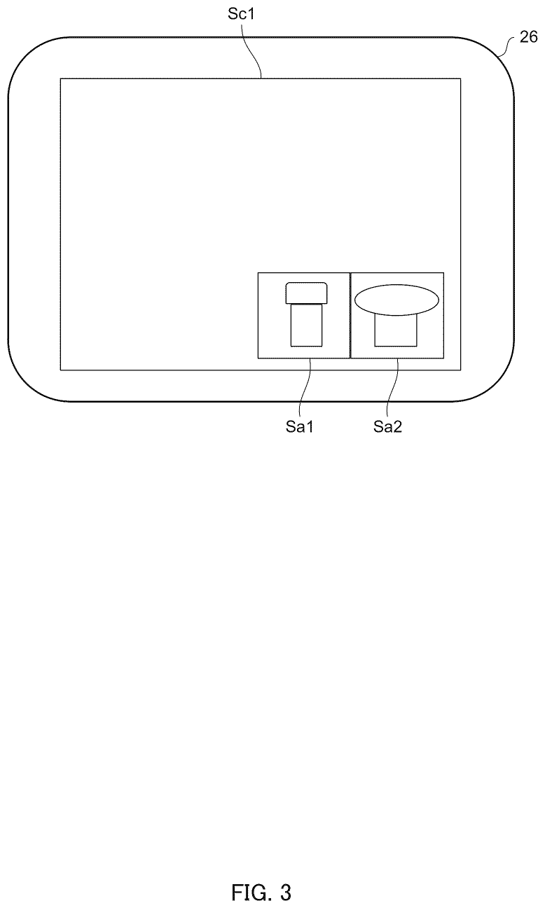

FIG. 2 is a flowchart illustrating an example of an ultrasound image processing method in the ultrasound image diagnostic system 100 (the ultrasound image processing apparatus 10 and the ultrasound image diagnostic apparatus 20). FIG. 3 is a view illustrating a selection screen Sc1 for selecting the ultrasound probe 30 in the display section 26 of the ultrasound image diagnostic apparatus 20.

(Step S11)

The type of the ultrasound probe 30 to be used for generating an ultrasound image is input to the control section 22 of the ultrasound image diagnostic apparatus 20. Note that at this time, a setting as to whether or not to perform automatic recognition to be described later may be input.

For example, the selection screen Sc1 shown in FIG. 3 is displayed on the display section 26 of the ultrasound image diagnostic apparatus 20 by the operation of the user (doctor or the like). The types of the ultrasound probe 30 that can be connected to the ultrasound image diagnostic apparatus 20 are displayed in the selection screen Sc1. Here, as an example, a sector probe and a convex probe can be connected to the ultrasound image diagnostic apparatus 20, and a selection region Sa1 for selecting the sector probe and a selection region Sa2 for selecting the convex probe are displayed on the selection screen Sc1. When the user selects one of the selection region Sa1 and the selection region Sa2 by using the operation section 27, the type of the ultrasound probe 30 is input to the control section 22.

In a case where the setting of whether to perform the automatic recognition is performed, a setting screen (not shown) for setting the automatic recognition is displayed on the display section 26 by the operation of the operator, and the operator sets the presence or absence of the automatic recognition using the operation section 27. Accordingly, the setting of the automatic recognition is input to the control section 22.

On the other hand, for example, in a case where the ultrasound image diagnostic apparatus 20 does not have an automatic recognition function, there may be no setting screen related to automatic recognition. Thus, the setting of whether to perform automatic recognition may be performed not on the ultrasound image diagnostic apparatus 20 side but on the ultrasound image processing apparatus 10 side. In this case, the setting of whether to perform the automatic recognition is performed on the ultrasound image processing apparatus 10 side before step S23 which will be described later. In addition, step S14 to be described later is also performed not on the ultrasound image diagnostic apparatus 20 side, but on the ultrasound image processing apparatus 10 side between step S23 and step S24.

(Step S12)

When a diagnosis is started by an operation of the operator, the control section 22 controls the signal transmission/reception section 23 and transmits and receives ultrasound waves to and from a subject with the ultrasound probe 30.

(Step S13)

The control section 22 controls the image generation section 24 to generate ultrasound image data as described above, based on reception signals from the ultrasound probe 30 that has received ultrasonic echoes.

(Step S14)

The control section 22 determines whether or not to perform automatic recognition, and in a case where the automatic recognition is to be performed (YES), the process proceeds to step S15, and in a case where the automatic recognition is not to be performed (NO), the process proceeds to step S18. When the setting of whether or not to perform the automatic recognition is input in advance in step S11, the control section 22 determines whether or not to perform the automatic recognition with reference to the setting. On the other hand, in a case where the setting as to whether or not the automatic recognition is performed is not input in advance, the control section 22 causes the display section 26 to display the setting screen for the automatic recognition such that the operator inputs the setting on the automatic recognition.

Note that as described above, when the setting as to whether or not to perform automatic recognition is performed on the ultrasound image processing apparatus 10 side, step S14 is performed not on the ultrasound image diagnostic apparatus 20 side but on the ultrasound image processing apparatus 10 side.

(Step S15)

The control section 22 controls the data transmission/reception section 25 such that the ultrasound image data and the discriminator determination information are transmitted to the ultrasound image processing apparatus 10. Here, processing in the ultrasound image processing apparatus 10 will be described.

(Step S21)

The control section 11 of the ultrasound image processing apparatus 10 checks whether a trained discriminator is present in the storage section 17. If the trained discriminator is present in the storage section 17 (YES), the process advances to step S23 to wait for the transmission of ultrasound image date and discriminator determination information from the ultrasound image diagnostic apparatus 20 described in step S15. When the trained discriminator is not present in the storage section 17 (NO), the process proceeds to step S22.

(Step S22)

As described above, the control section 11 performs training with teacher data using the teacher data generation section 19 and the learning section 18 to generate a discriminator. The generation of the discriminator will be described below by taking an exemplary case of automatic measurement of the diameter of inferior vena cava (hereinafter, referred to as IVC) and an exemplary case of automatic classification of measurement items in obstetrics.

Note that as described above, when the setting as to whether or not to perform the automatic recognition is performed on the ultrasound image processing apparatus 10 side, the setting is performed before step S23, for example, before step S21 or between step S22 and step S23. In this case, the setting screen (not shown) for automatic recognition is displayed on the display section 15 by the operation of the operator, and the operator sets the presence or absence of automatic recognition using the operation section 16, whereby the setting of automatic recognition is input to the control section 11.

(Step S23)

The control section 11 receives the ultrasound image data and discriminator determination information from the ultrasound image diagnostic apparatus 20 using the data transmission/reception section 12.

Note that as described above, when the setting as to whether or not to perform automatic recognition is performed on the ultrasound image processing apparatus 10 side, the control section 11 makes a determination similar to that in step S14 described above between step S23 and step S24. That is, the control section 11 determines, according to the setting on the ultrasound image processing apparatus 10 side, whether to perform the automatic recognition. When the automatic recognition is to be performed (YES), the process proceeds to step S24, and when the automatic recognition is not to be performed (NO), the process proceeds to step S18.

(Step S24)

The control section 11 sets, by using the determination section 13, the discriminator based on the discriminator determination information received from the ultrasound image diagnostic apparatus 20.

(Step S25)

The control section 11 inputs the ultrasound image data received from the ultrasound image diagnostic apparatus 20 to the discriminator set in step S24, executes inference by the automatic recognition, and acquires a discrimination result using the acquisition section 14.

(Step S26)

The control section 11 transmits the discrimination result acquired in step S25 to the ultrasound image diagnostic apparatus 20 using the data transmission/reception section 12.

A description will be given with reference again to the processing in the ultrasound image diagnostic apparatus 20.

(Step S16)

The control section 22 receives the discrimination result from the ultrasound image processing apparatus 10 using the data transmission/reception section 25.

(Step S17)

The control section 22 uses the display section 26 such that the display section 26 displays the ultrasound image generated by the image generation section 24 and the discrimination result from the ultrasound image processing apparatus 10. That is, in a case in which the automatic recognition is performed, the control section 22 displays the ultrasound image corresponding to the ultrasound image data generated by the image generation section 24 and the discrimination result from the ultrasound image processing apparatus 10 using the display section 26.

As described above, since the discrimination result is transmitted from the ultrasound image processing apparatus 10 to the ultrasound image diagnostic apparatus 20 and the ultrasound image and the discrimination result are displayed using the display section 26, it is possible to perform diagnosis on the ultrasound image diagnostic apparatus 20 side. Similarly, in the ultrasound image processing apparatus 10, the ultrasound image and the discrimination result may be displayed using the display section 15, and diagnosis may be performed in the ultrasound image processing apparatus 10.

(Step S18)

The control section 22 uses the display section 26 such that the display section 26 displays the ultrasound image corresponding to the ultrasound image data generated by the image generation section 24. That is, in the case in which the automatic recognition is not performed, the control section 22 causes, to be displayed, the ultrasound image generated by the image generation section 24.

[Ultrasound Image Processing Method—Case of Automatic Measurement of IVC Diameter]

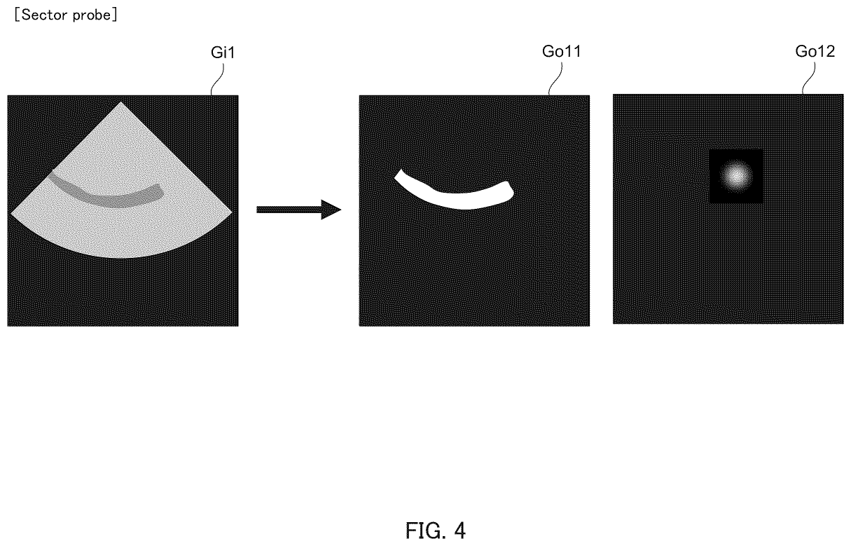

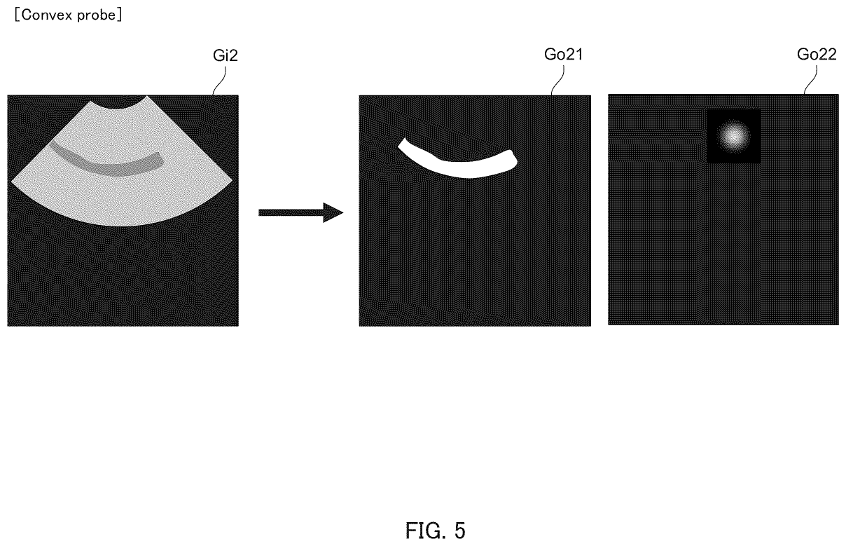

FIG. 4 is a view illustrating an ultrasound image of teacher data acquired using a sector probe, and an IVC region and a hepatic vein position to be automatically recognized by a discriminator. FIG. 5 is a view illustrating an ultrasound image of teacher data acquired using a convex probe, and an IVC region and a hepatic vein position to be automatically recognized by a discriminator.

In the above-described ultrasound image processing method, in a case where the automatic measurement of the IVC diameter is performed, the following learning is performed in step S22 which is a training phase.

(1) Case of Sector Probe

The teacher data generation section 19 generates, as teacher data, ultrasound image data which is acquired by the ultrasound image diagnostic apparatus 20 using a sector probe and which includes information indicating an IVC region and a hepatic vein position. For example, the teacher data generation section 19 generates an ultrasound image Gi1 of the teacher data illustrated in FIG. 4. The learning section 18 generates the discriminator 1 by learning the teacher data generated by the teacher data generation section 19 so as to automatically recognize the IVC region and the hepatic vein position. For example, the learning section 18 recognizes the IVC region (white portion) shown in the image Go11 of FIG. 4 or the hepatic vein position (white portion) shown in the image Go12.

(2) Case of Convex Probe

The teacher data generation section 19 generates, as teacher data, ultrasound image data which is acquired by the ultrasound image diagnostic apparatus 20 using a convex probe and which includes information indicating the IVC region and the hepatic vein position. For example, the teacher data generation section 19 generates an ultrasound image Gi2 of the teacher data illustrated in FIG. 5. The learning section 18 generates the discriminator 2 by learning the teacher data generated by the teacher data generation section 19 so as to automatically recognize the IVC region and the hepatic vein position. For example, the learning section 18 recognizes the IVC region (white portion) shown in the image Go21 of FIG. 5 or the hepatic vein position (white portion) shown in the image Go22.

In the storage section 17 of the ultrasound image processing apparatus 10, the discriminator 1 and the discriminator 2 are stored in association with the types of the ultrasound probe 30.

Next, in the above-described ultrasound image processing method, when the automatic measurement of the IVC diameter is performed, the following inference is performed in the above-described steps S24 and S25 that are the inference phase.

Based on the discriminator determination information transmitted from the ultrasound image diagnostic apparatus 20, the determination section 13 sets the discriminator to be used for the transmitted ultrasound image data. In a case where the discriminator determination information is the sector probe, the discriminator 1 is set, and in a case where the discriminator determination information is the convex probe, the discriminator 2 is selected (step S24).

When the acquisition section 14 inputs the ultrasound image data to the set discriminator, the discriminator performs automatic recognition processing on the ultrasound image data. The discriminator determines an IVC region and a hepatic vein position in the ultrasound image data, determines two points for measuring an IVC diameter using the determined IVC region and hepatic vein position, and calculates a distance between the determined two points. The discriminator outputs, for example, the determined positions (regions of interest) of two points and the calculated distance (automatic measurement results) as a discrimination result (step S25).

[Ultrasound Image Processing Method—Case of Automatic Classification of Measurement Items in Obstetrics]

In the ultrasound image processing method described above, in a case where automatic classification of measurement items in obstetrics is performed, the following training is performed in step S22 described above that is the training phase.

(1) In Case of Intracavitary Probe

The teacher data generation section 19 generates, as teacher data, measurement items of a Crown-Rump Length (hereinafter referred to as CRL) and a Bi-Parietal Diameter (hereinafter referred to as BPD) in ultrasound image data acquired by the ultrasound image diagnostic apparatus 20 using an intracavitary probe. For the purpose of automatic classification of the measurement items by the intracavitary probe, the learning section 18 learns the teacher data generated by the teacher data generation section 19 and generates the discriminator 3 such that the measurement items of the CRL and the BPD are automatically recognized and the measurement position for each measurement item is automatically recognized

(2) Case of Convex Probe

The teacher data generation section 19 generates, as teacher data, measurement items of the CRL, the BPD, an Abdominal Circumference (hereinafter referred to as AC), and a Femur Length (FL) in ultrasound image data acquired by the ultrasound image diagnostic apparatus 20 using a convex probe. For the purpose of automatic classification of measurement items by the convex probe, the learning section 18 learns the teacher data generated by the teacher data generation section 19 and generates the discriminator 4 such that the measurement items of the CRL, BPD, AC, and FL are automatically recognized and the measurement position for each measurement item.

The storage section 17 of the ultrasound image processing apparatus 10 stores the discriminator 3 and the discriminator 4 in association with the types of the ultrasound probe 30.

Next, in the case of performing automatic classification of measurement items in obstetrics in the above-described ultrasound image processing method, the following inference is performed in the above-described steps S24 and S25 that are the inference phase.

The determination section 13 sets the discriminator to be used for the transmitted ultrasound image data based on the discriminator determination information transmitted from the ultrasound image diagnostic apparatus 20. In a case where the discriminator determination information indicates the intracavitary probe, the discriminator 3 is set, and in a case where the discriminator determination information indicates the convex probe, the discriminator 4 is selected (step S24).

When the acquisition section 14 inputs the ultrasound image data to the set discriminator, the discriminator performs automatic recognition processing on the ultrasound image data. The discriminator decides a measurement item in the ultrasound image data, decides a measurement position corresponding to the decided measurement item, and calculates a measurement value of the decided measurement position. The discriminator outputs, as the discrimination result, for example, the determined measurement position (region of interest) and the measurement value (automatic measurement result) calculated correspondingly to the measurement item (classification result of the measurement item) (step S25).

As described above, in the ultrasound image diagnostic apparatus 20, the ultrasound image data acquired by different ultrasound probes 30 for the same part of the subject is used as the teacher data, and the discriminator is generated for each of the ultrasound probes 30. That is, the discriminator is prepared for each of the different ultrasound probes 30 that can be connected to the ultrasound image diagnostic apparatus 20. Next, since a corresponding discriminator is set based on the discriminator determination information transmitted from the ultrasound image diagnostic apparatus 20, automatic recognition on ultrasound image data can be performed using an optimal discriminator, and a highly accurate discrimination result can be acquired.

In addition, for an ultrasound image diagnostic apparatus 20 manufactured by a different manufacturer, discriminators may be prepared respectively for different ultrasound probes 30 connectable to the ultrasound image diagnostic apparatus 20 as described above. Accordingly, since a corresponding discriminator is set based on the discriminator determination information transmitted from the ultrasound image diagnostic apparatus 20 manufactured by the different manufacturer, automatic recognition on ultrasound image data can be performed using an optimal discriminator, and a highly accurate discrimination result can be acquired.

As described above, in the present embodiment, the ultrasound image processing apparatus 10 includes the data transmission/reception section 12, the determination section 13, and the acquisition section 14. The data transmission/reception section 12 receives the ultrasound image data and the discriminator determination information from the ultrasound image diagnostic apparatus 20. Based on the discriminator determination information received from the ultrasound image diagnostic apparatus 20, the determination section 13 determines a discriminator to which the ultrasound image data is to be input, from among a plurality of discriminators that discriminate the discrimination target in the ultrasound image data. The acquisition section 14 inputs the ultrasound image data to the discriminator to acquire a discrimination result output from the discriminator.

In this embodiment, the ultrasound image diagnostic system 100 includes the ultrasound image processing apparatus 10 and the ultrasound image diagnostic apparatus 20. The ultrasound image diagnostic apparatus 20 includes the ultrasound probe 30, the image generation section 24, and the data transmission/reception section 25. The ultrasound probe 30 transmits and receives ultrasound waves to and from a subject. The image generation section 24 generates the ultrasound image data based on the reception signal acquired by the ultrasound probe 30. The data transmission/reception section 25 transmits the ultrasound image data and the discriminator determination information to the ultrasound image processing apparatus 10.

According to the ultrasound image processing apparatus 10 and the ultrasound diagnostic imaging system 100 of the present embodiment configured as described above, it is possible to acquire a discrimination result by automatic recognition even for ultrasound image data acquired by the ultrasound image diagnostic apparatus 20 which does not have an automatic recognition function (discriminator). Therefore, even when remote ultrasound diagnosis is performed using a different ultrasound probe 30 and/or a different ultrasound image diagnostic apparatus 20, automatic recognition can be performed more appropriately, and as a result, the work load on an operator (doctor or the like) can be reduced.

According to the ultrasound image processing apparatus 10 and the ultrasound image diagnostic system 100 of this embodiment, the discriminators are prepared for the different ultrasound probes 30 that can be connected to the ultrasound image diagnostic apparatus 20. In addition, since an optimal discriminator is set based on the discriminator determination information received from the ultrasound image diagnostic apparatus 20, the operator can perform automatic recognition using any of the ultrasound probes 30, and can acquire a highly accurate discrimination result.

The present embodiment is particularly useful in an environment in which diagnosis is performed at a remote location using a portable ultrasound image diagnostic apparatus 20, and even in a case where the portable ultrasound image diagnostic apparatus 20 does not have an automatic recognition function (discriminator), it is possible to acquire a discrimination result by automatic recognition.

In addition, in a case where discriminators are prepared respectively for different ultrasound probes 30 which can be connected to the ultrasound image diagnostic apparatuses 20 of different manufacturers, it is possible to acquire a discrimination result by automatic recognition even in a case where diagnosis is performed using the ultrasound image diagnostic apparatuses 20 of different manufacturers.

<Variation 1>

In the above-described embodiment, the ultrasound image processing apparatus 10 includes the discriminator. In the present variation, in addition to the ultrasound image processing apparatus 10, the ultrasound image diagnostic apparatus 20 also includes the above-described discriminator.

In the present variation, basically, the ultrasound image diagnostic apparatus 20 performs automatic recognition on acquired ultrasound image data using the discriminator. Meanwhile, for example, if the processing capability of the CPU of the control section 22 is not high or the storage capacity of the storage section 28 is not large in the ultrasound image diagnostic apparatus 20, it is difficult to use a plurality of discriminators. In such a case, in addition to setting of automatic recognition, determination of whether to proceed to step S15 or step S18 may be made in step S14 described above based on the discriminator determination information (a determination section according to the present invention). That is, the control section 22 may determine, based on the discriminator determination information, whether or not to transmit the ultrasound image data and the discriminator determination information to the ultrasound image processing apparatus 10.

In this case, the control section 22 checks, based on the discriminator determination information, whether the discriminator corresponding to the discriminator determination information is present in the storage section 28, and proceeds to step S18 in a case where the discriminator corresponding to the discriminator determination information is present. On the other hand, when there is not the discriminator corresponding to the discriminator determination information, the process proceeds to step S15, and automatic recognition on the ultrasound image is performed using the discriminator stored in the ultrasound image processing apparatus 10.

As described above, in a case where a discriminator suitable for the type of the ultrasound probe 30 in use is present on the ultrasound image diagnostic apparatus 20 side, it is possible not to transmit the ultrasound image data and the discriminator determination information to the ultrasound image processing apparatus 10.

On the other hand, when the ultrasound image diagnostic apparatus 20 does not have the discriminator suitable for the type of the ultrasound probe 30 in use, the ultrasound image data and the discriminator determination information are transmitted to the ultrasound image processing apparatus 10, and thus it is possible to acquire a discrimination result by automatic recognition. Therefore, even when remote ultrasound diagnosis is performed using a different ultrasound probe 30 and/or a different ultrasound image diagnostic apparatus 20, automatic recognition can be performed more appropriately, and as a result, the work load on an operator (doctor or the like) can be reduced.

<Other Variations>

The above-described embodiment and variations are merely examples for implementing the present invention, and should not be construed as limiting the technical scope of the present invention. In other words, the present invention can be embodied in various forms without departing from the spirit, scope, or principal features of the present invention.

For example, the discriminator determination information is not limited to information on the ultrasound probe 30, and may be at least one of information on the ultrasound probe 30, information on an ultrasound image, information on a discriminator, and information on the ultrasound image diagnostic apparatus 20.

The information on the ultrasound image diagnostic apparatus 20 is, for example, information on a manufacturer which manufactures the ultrasound image diagnostic apparatus 20. By using the information on the manufacturer as the discriminator determination information, a discriminator optimal for the image quality of an ultrasound image for each manufacturer can be selected, and by performing inference using the optimal discriminator, a highly accurate discrimination result can be acquired.

The information on the ultrasound image may include information on a depth of field or a sampling interval of the ultrasound image. By using the information on the depth of field or the sampling interval of the ultrasound image, the measurement of the distance between two points and the like can be calculated in actual size.

The information on the ultrasound image may be transmitted by attaching a predetermined file (xml file, csv file, or the like) to the ultrasound image or the raw data of the ultrasound image. In addition, at the time of transmission, the information may be attached to a communication header, and for example, may be transmitted in the same manner as a header of Digital Imaging and Communications in Medicine (DICOM).

Furthermore, the discriminator determination information may be information on a part of a subject or a medical department. For the automatic recognition on an ultrasound image, there are cases where items of automatic recognition to be performed are different or there are a wide variety of options for each medical department such as obstetrics, cardiology, and orthopedics or for each site such as upper limb, abdomen, and fetus. Even in such cases, the discriminator is selected based on the discriminator determination information described above. Thus, the items of automatic recognition are narrowed down, and in a case where selection of automatic recognition is necessary, the time and effort required for the selection can be reduced. Further, in a case where the items of the automatic recognition are to be automatically distinguished, an incorrect option is excluded in advance. Accordingly, an automatic recognition result with higher accuracy is acquired.

When there is no discriminator determination information transmitted from the ultrasound image diagnostic apparatus 20, the ultrasound image processing apparatus 10 may perform automatic recognition on an ultrasound image using a general-purpose discriminator. For example, some ultrasound image diagnostic apparatuses 20 cannot transmit the discriminator determination information. As described above, even in an ultrasound image acquired by the ultrasound image diagnostic apparatus 20 which cannot transmit discriminator determination information, desired measurement and diagnosis can be performed by performing automatic recognition using the general-purpose discriminator.

In a case where there is no discriminator determination information transmitted from the ultrasound image diagnostic apparatus 20, the ultrasound image processing apparatus 10 may analyze the ultrasound image data, acquire the discriminator determination information, and determine an optimal discriminator based on the acquired discriminator determination information.

For example, since the image size, the frame rate, and the image quality that can be rendered in the ultrasound image are different depending on a difference in the type of the ultrasound probe 30, the discriminator determination information on the type of the ultrasound probe 30 can be acquired, and the optimal discriminator can be determined based on the acquired discriminator determination information.

Therefore, even in the case of ultrasound image data acquired by the ultrasound image diagnostic apparatus which cannot transmit discriminator determination information, automatic recognition is performed using an optimal discriminator, and a highly accurate discrimination result can be acquired in desired measurement or diagnosis.

Although embodiments of the present invention have been described and illustrated in detail, the disclosed embodiments are made for purpose of illustration and example only and not limitation. The scope of the present invention should be interpreted by terms of the appended claims

What is claimed is:

1. An ultrasound image processing apparatus, comprising:
a receiver that receives an ultrasound image and discriminator determination information from an external apparatus, the ultrasound image being generated based on a reception signal acquired by an ultrasound probe that transmits and receives an ultrasound wave to and from a subject, the discriminator determination information allowing determination of a discriminator that discriminates a target in the ultrasound image; and
one or more first hardware processors, wherein
the one or more first hardware processors determine, from among a plurality of the discriminators, a discriminator to which the ultrasound image is to be input, the discriminator being determined based on the discriminator determination information received from the external apparatus, and
the one or more first hardware processors input the ultrasound image to the determined discriminator to acquire a discrimination result output from the determined discriminator.

2. The ultrasound image processing apparatus according to claim 1, wherein
the discriminator determination information is at least one of information on the ultrasound probe, information on the ultrasound image, information on the discriminator, and information on the external apparatus.

3. The ultrasound image processing apparatus according to claim 2, wherein
the discriminator determination information is the information on the external apparatus, and the information on the external apparatus is information on a manufacturer which manufactures the external apparatus.

4. The ultrasound image processing apparatus according to claim 1, wherein
the discrimination result is at least one of a classification result of a measurement item, an automatic measurement result, and a region of interest.

5. An ultrasound image diagnostic system, comprising:
an ultrasound image acquisition apparatus; and
an ultrasound image processing apparatus, wherein:
the ultrasound image acquisition apparatus includes:
an ultrasound probe that transmits and receives an ultrasound wave to and from a subject,
a generator that generates an ultrasound image based on a reception signal acquired by the ultrasound probe, and
a transmitter that transmits, to the ultrasound image processing apparatus, the ultrasound image and discriminator determination information allowing determination of a discriminator that discriminates a target in the ultrasound image,
the ultrasound image processing apparatus includes:
a receiver that receives the ultrasound image and the discriminator determination information,
a plurality of discriminators that discriminate the target in the ultrasound image, and
one or more first hardware processors,
the one or more first hardware processors determine, from among the plurality of the discriminators, a discriminator to which the ultrasound image is to be input, the discriminator being determined based on the discriminator determination information, and
the one or more first hardware processors input the ultrasound image to the determined discriminator to acquire a discrimination result output from the determined discriminator.

6. The ultrasound image diagnostic system according to claim 5, wherein:
the ultrasound image processing apparatus includes a transmitter that transmits the discrimination result to the ultrasound image acquisition apparatus, and
the ultrasound image acquisition apparatus includes a receiver that receives the discrimination result from the ultrasound image processing apparatus.

7. The ultrasound image diagnostic system according to claim 5, wherein
the ultrasound image acquisition apparatus includes at least one discriminator that discriminates the target in the ultrasound image.

8. The ultrasound image diagnostic system according to claim 5, wherein:
the ultrasound image acquisition apparatus includes one or more second hardware processors, and
the one or more second hardware processors determine based on the discriminator determination information whether or not to transmit the ultrasound image and the discriminator determination information to the ultrasound image processing apparatus.

9. An ultrasound image processing method, comprising:
receiving an ultrasound image and discriminator determination information from an external apparatus, the ultrasound image being generated based on a reception signal acquired by an ultrasound probe that transmits and receives an ultrasound wave to and from a subject, the discriminator determination information allowing determination of a discriminator that discriminates a target in the ultrasound image;
determining, from among a plurality of the discriminators, a discriminator to which the ultrasound image is to be input, the discriminator being determined based on the discriminator determination information received from the external apparatus; and
inputting the ultrasound image to the determined discriminator to acquire a discrimination result output from the determined discriminator.

10. A non-transitory computer-readable recording medium storing an ultrasound image processing program for causing a computer to execute:
a process of receiving an ultrasound image and discriminator determination information from an external apparatus, the ultrasound image being generated based on a reception signal acquired by an ultrasound probe that transmits and receives an ultrasound wave to and from a subject, the discriminator determination information allowing determination of a discriminator that discriminates a target in the ultrasound image,
a process of determining, from among a plurality of the discriminators, a discriminator to which the ultrasound image is to be input, the discriminator being determined based on the discriminator determination information received from the external apparatus; and
a process of inputting the ultrasound image to the determined discriminator to acquire a discrimination result output from the determined discriminator.

11. The non-transitory computer-readable recording medium storing the ultrasound image processing program according to claim 10, wherein
the discriminator determination information is at least one of information on the ultrasound probe, information on the ultrasound image, information on the discriminator, and information on the external apparatus.

12. The non-transitory computer-readable recording medium storing the ultrasound image processing program according to claim 11, wherein
the discriminator determination information is the information on the external apparatus, and the information on the external apparatus is information on a manufacturer which manufactures the external apparatus.

13. The non-transitory computer-readable recording medium storing the ultrasound image processing program according to claim 10, wherein
the discrimination result is at least one of a classification result of a measurement item, an automatic measurement result, and a region of interest.
